(19) United States
(12) Patent Application Publication
(10) Pub. No.: US 2013/0012960 A1
(43) Pub. Date: Jan. 10, 2013

(54) SURGICAL SUTURE CUTTING DEVICE

(76) Inventor: YEN-YUE LIN, Zhongli City (TW)

(21) Appl. No.: 13/490,469

(22) Filed: Jun. 7, 2012

(30) Foreign Application Priority Data
Jul. 8, 2011 (TW) 100124194

(51) Int. Cl. A61B 17/10

(52) U.S. Cl. 606/138

(57) ABSTRACT

The surgical suture cutting device contains a body having two bulging sections extended from a front end of the body, forming a notch in between. A knife is wrapped inside the body whose blade is exposed in the notch. With the blade and the bulging sections, the surgical suture cutting device can be operated with a single hand to perform both tissue pressing and suture cutting. In addition, since the blade is protected in the notch, the patient will not be mistakenly wounded. The bulging sections also provide comfortable tissue pressing. Therefore, the surgical suture cutting device greatly enhances the efficiency of the subcutaneous stitching-up operation. A simple knife wrapped in the body also allows simple and inexpensive production.

### TECHNICAL FIELD OF THE INVENTION

The present invention is generally related to medical suture cutting devices, and more particularly to a medical suture cutting device especially adapted for subcutaneous stitching-up operation that is safe to operate, and easy to manufacture at a lower cost.

### DESCRIPTION OF THE PRIOR ART

In stitching up a wound, the doctor usually holds a forceps in one hand to hold the patient's tissue together, and uses the other hand to operate a needle holder. After each stitch, the doctor has to tie the suture, then empties a hand first, and operates a scissors to cut the suture. This is a cumbersome process even with a nurse to help cutting the suture.

Therefore a dual-function forceps providing both cutting and holding has been taught. The forceps is actually a tweezers with a detachable cutter at one end. Therefore, the doctor is able to use a single hand to do both suture cutting and tissue holding. There is no need to switch to a scissors or to ask nursing staff to help.

The dual-function forceps, however, has the following disadvantages.

Firstly, a blade of the cutter is configured in a notch between two bulging curved sections along a top rim of the cutter. The blade is made of metal and has a complex shape so that it is not appropriate to form using stamping or polishing, thereby leading to a higher production cost.

As a matter of fact, cutting the suture only requires some sharp edge. There is no need to manufacture a metallic blade.

Additionally, the blade does not have proper protection mechanism and there is some potential hazard that the patient might be wounded. For example, in the subcutaneous stitching-up operation, the tissue around the wound has to be pressed so as to pull, tie, and cut the suture. In the process, not only the patient would feel uncomfortable, but also the unprotected blade might hurt the patient.

### SUMMARY OF THE INVENTION

Therefore, a novel surgical suture cutting device is provided here that is adapted for subcutaneous stitching-up operation, and easy and inexpensive to manufacture.

The surgical suture cutting device contains a body having two bulging sections extended from a front end of the body, forming a notch in between. A knife is wrapped inside the body whose blade is exposed in the notch, With the blade and the bulging sections, the surgical suture cutting device can be operated with a single hand to perform both tissue pressing and suture cutting. In addition, since the blade is protected in the notch, the patient will not be mistakenly wounded. The bulging sections also provide comfortable tissue pressing. Therefore, the surgical suture cutting device greatly enhances the efficiency of the subcutaneous stitching-up operation. A simple knife wrapped in the body also allows simple and inexpensive production.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a schematic perspective diagram showing a surgical suture cutting device according to a first embodiment of the present invention.

FIG. 2 is a schematic top-view diagram showing the surgical suture cutting device of FIG. 1.

FIG. 3 is a perspective side-view diagram showing the surgical suture cutting device of FIG. 1.

FIG. 4 is a schematic rear-view diagram showing the surgical suture cutting device of FIG. 1.

FIG. 5 is a schematic perspective diagram showing a tweezers joined to the surgical suture cutting device of FIG. 1 with a schematic sectional view showing the interaction of the tweezers and surgical suture cutting device.

FIG. 6 is a schematic top-view diagram showing a tweezers joined to the surgical suture cutting device of FIG. 1.

FIG. 7 is a schematic side-view diagram showing a tweezers joined to the surgical suture cutting device of FIG. 1.

FIG. 8 is a perspective diagram showing how the surgical suture cutting device of FIG. 1 is applied in the subcutaneous stitching-up operation.

FIG. 9 is a schematic perspective diagram showing a surgical suture cutting device according to a second embodiment of the present invention.

FIG. 10 is a schematic rear-view diagram showing the surgical suture cutting device of FIG. 9.

FIG. 11 is a schematic perspective diagram showing a surgical suture cutting device according to a third embodiment of the present invention.

FIG. 12 is a schematic side-view diagram showing a surgical suture cutting device according to a fourth embodiment of the present invention.

FIG. 13 is a schematic side-view diagram showing a tweezers of an ordinary thickness plugged into the surgical suture cutting device of FIG. 12.

FIG. 14 is a schematic side-view diagram showing a thick tweezers plugged into the surgical suture cutting device of FIG. 12.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

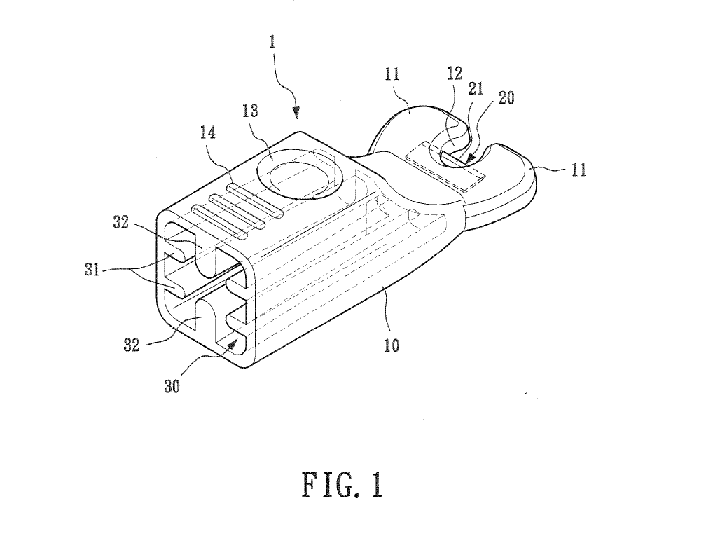

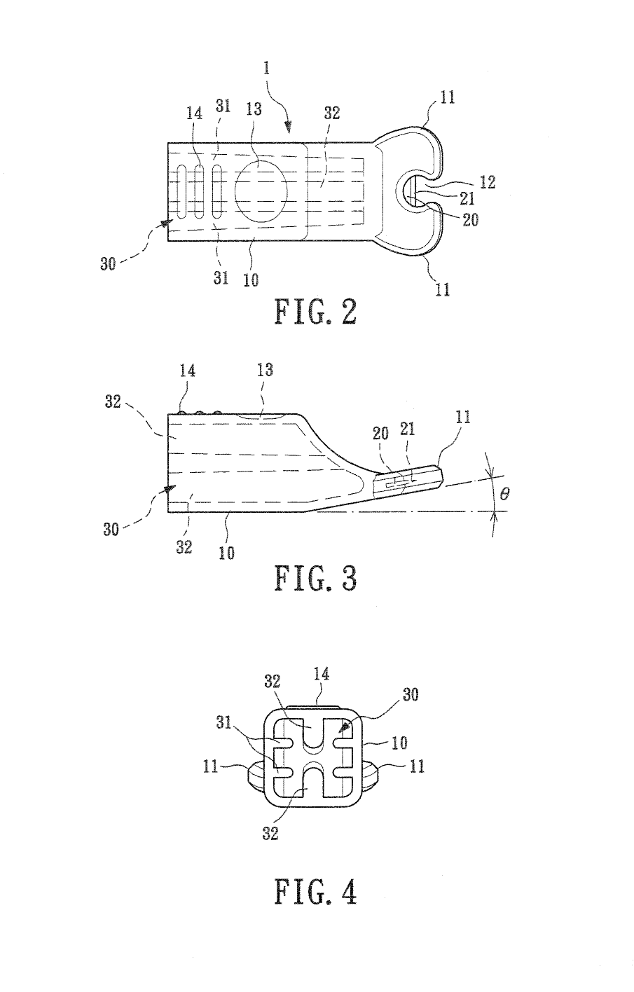

As shown in FIGS. 1 to 4, a surgical suture cutting device 1 according to a first embodiment of the present invention contains a rectangular body 10, a knife 20 embedded in the body 10, and a coupling section 30. The surgical suture cutting device 1 can be used independently or, after joining a tweezers or forceps to the coupling section 30, the assembly can be operated with a single hand to perform both tissue pressing and suture cutting without mistakenly wounding the patient. A simple knife 20 wrapped in the body 10 also allows simple and inexpensive production.

The body 10 is integrally formed with materials like plastic or rubber. The body 10 has two curved bulging sections 11 extended in parallel from a front end, thereby forming a notch 12 in between. On a top side of the body 10, there are an indentation 13 and a number of ribs 14, so as to enhance the friction for easier and anti-slippery operation with fingers. They also add to the appeal of the appearance. The bulging sections 11 are tilted upward at an angle θ with a bottom side of the body 10. As such, the operation of the surgical suture cutting device 1 is more ergonomic and convenient.

The knife 20 is a slim rectangular piece and is partly wrapped inside the body 10, and a blade 21 of the knife 20 is exposed outside the body 10 in the notch 12.

The coupling section 30 at a rear end of body 10 is an integral part of the body 10. The coupling section 30 provides a rectangular chamber open to the rear end. From each of the chamber's two opposing lateral walls, two thin positioning ribs 31 are extended in parallel. From each of the chamber's top and bottom walls, a thick positioning rib 32 is extended.

Compared to the prior arts, the surgical suture cutting device described above has the following advantages.

Firstly, the bulging sections 11 and the notch 12 are integrally formed, and the knife 20 is of a simple shape that can be easily produced with stamping or polishing. Therefore, the production cost of the surgical suture cutting device can be significantly reduced.

Secondly, the body 10 is made of plastic or rubber and the knife, even though it is metallic, is reduced to the minimum. As such, the production of the surgical suture cutting device's cost can be maintained at a low level.

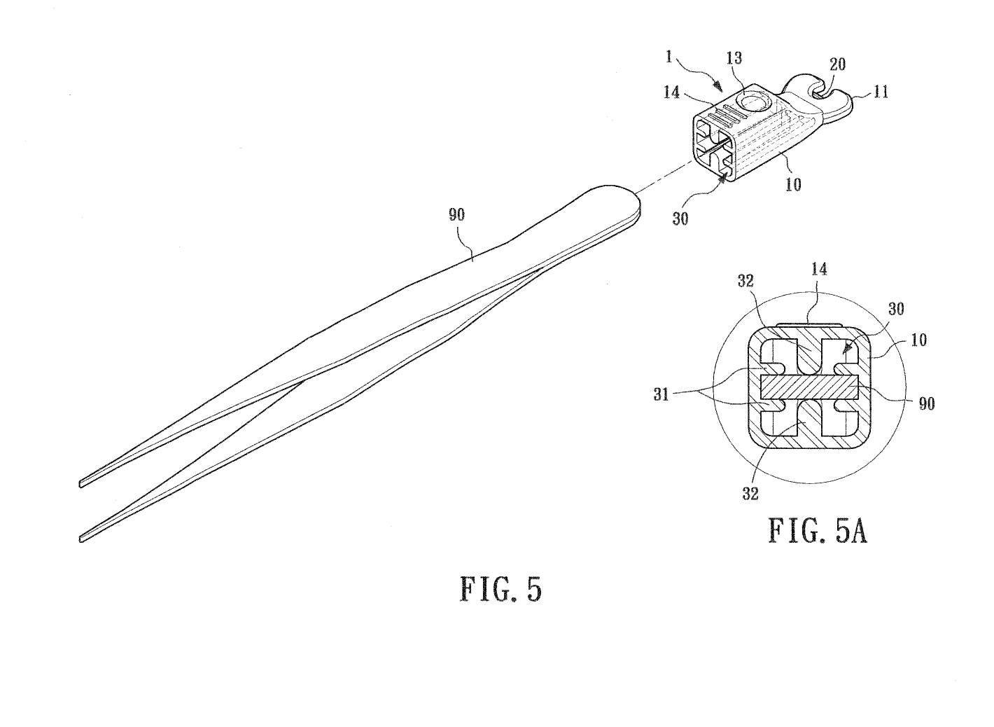

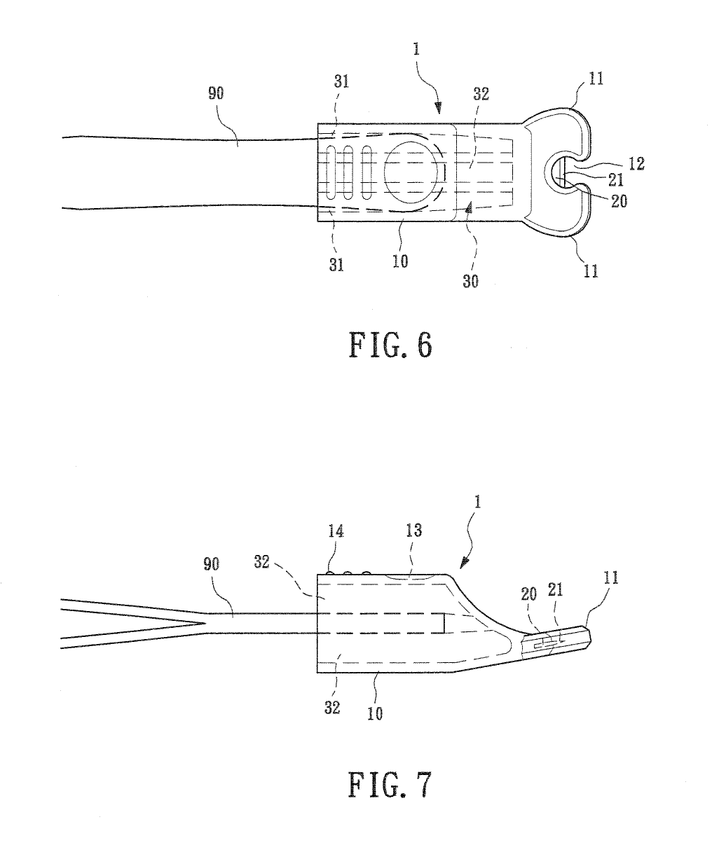

As shown in FIGS. 5 to 7, a tweezers (or forceps) 90 can be joined to the coupling section 30 by plugging the flat and close end of the tweezers 90 laterally into the chamber of the coupling section 30. The lateral edges of the flat and close end of the tweezers 90 are sandwiched between the two pairs of the think positioning ribs 31, respectively. The downward and upward extended thick positioning ribs 32 press against the top and bottom sides of the flat and close end of the tweezers 90, respectively. As such, the tweezers 90 is tightly and reliably held by the coupling section 30. When not in use, the tweezers 90 can also be removed from the coupling section 30 easily for easy storage and carrying.

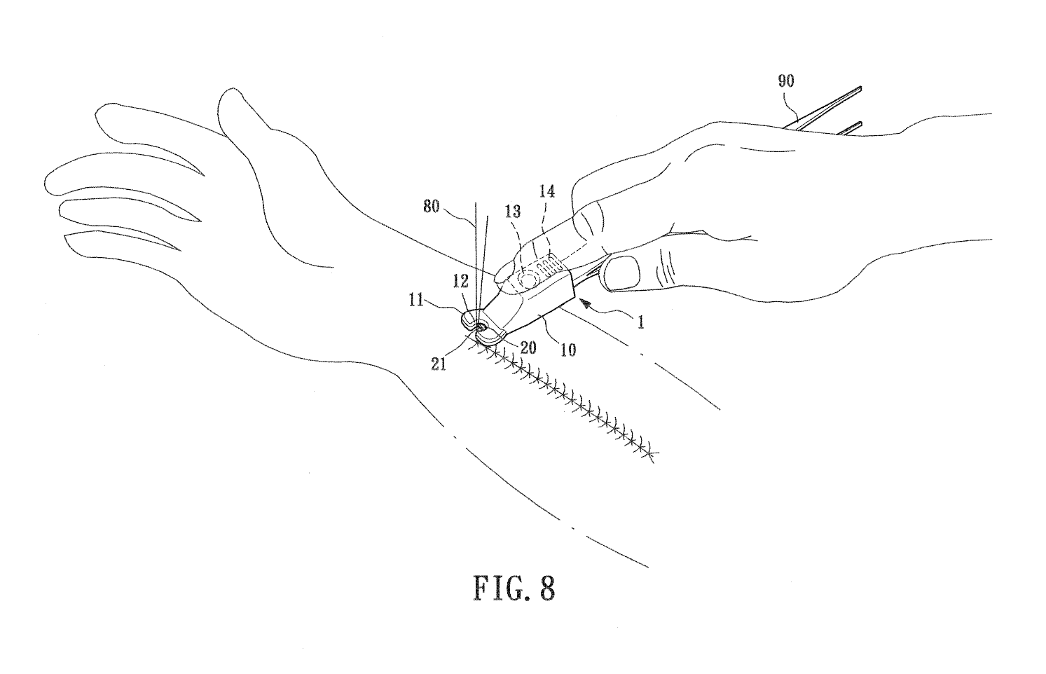

FIG. 8 shows how the present embodiment is utilized in a subcutaneous stitching-up operation to cut the surgical suture. After the tweezers 90 is joined to the surgical suture cutting device 1, the doctor can switch between the tweezers 90 and the knife 20 at will. After stitching up the wound and the suture 80 is tie, the doctor can use the bulging sections 11 to press down the tissue around the wound and pull out the knot tied by the suture 80. Then, by arranging the suture 80 in the notch 12 and pushing the blade 21 forward, the superfluous suture 80 can be cut down. The doctor does need to use a scissors or the help of the others.

Please note that, by pressing the tissue with the bulging sections 11, the doctor can accomplish the tissue pressing and suture cutting with a single hand. Additionally, as the blade 221 is isolated in the notch 12 to prevent the patient from being accidentally wounded. The curved shape of the bulging sections 11 also provides safe contact and comfortable feeling to the patient. The surgical suture cutting device is therefore especially suitable for subcutaneous stitching-up operation.

Furthermore, as the bulging sections 11 are tilted at an angle θ and when the bulging sections 11 are flatly pressed against the skin, the body 10 and the tweezers 90 are tilted upward for better ergonometric and more convenient operation. The indentation 13 and the ribs 14 provide greater friction and the surgical suture cutting device is thereby easier and more reliable to handle.

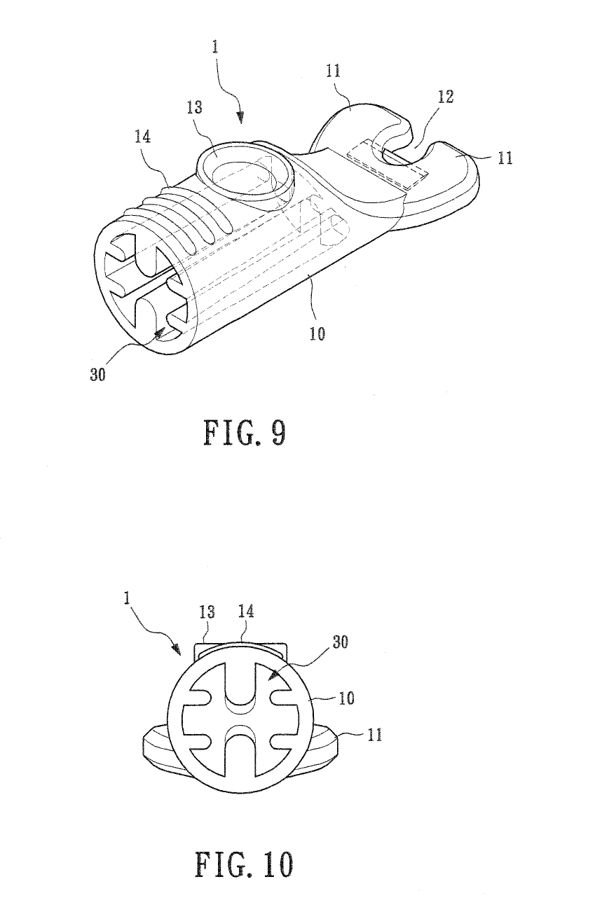

As shown in FIGS. 9 and 10, a surgical suture cutting device 1 according to a second embodiment of the present invention has a cylindrical body 10, and the chamber provided by the coupling section 30 has a circular cross-section.

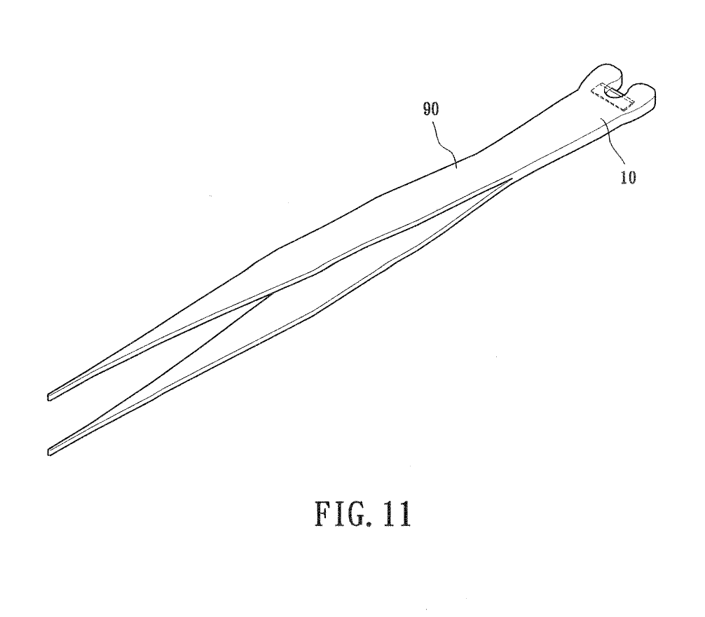

As shown in FIG. 11, a surgical suture cutting device 1 according to a third embodiment of the present invention has a body 10 and the tweezers 90 integrally formed together.

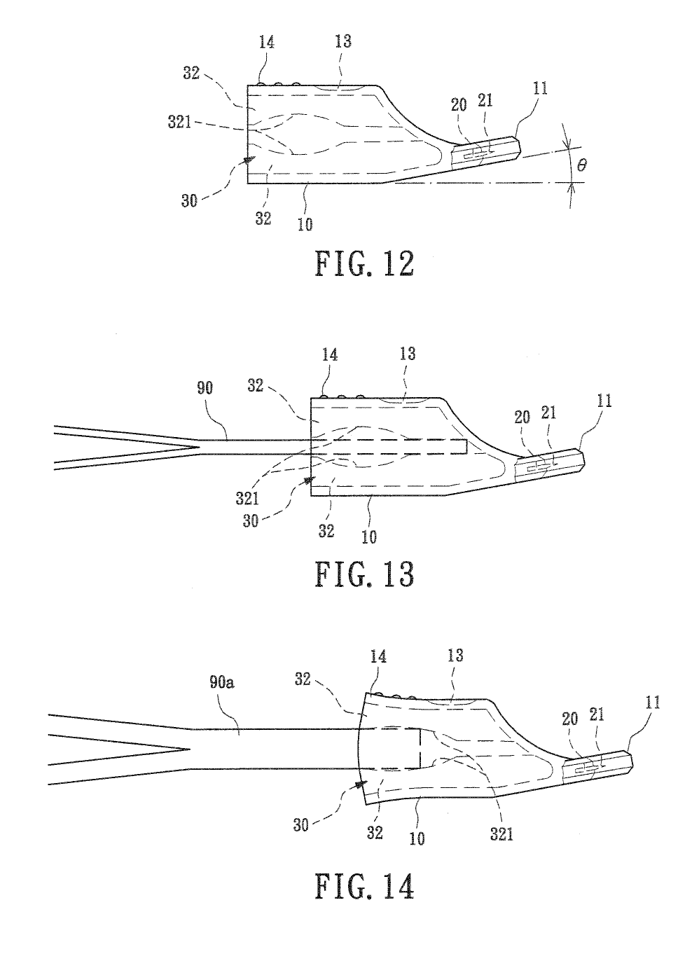

As shown in FIGS. 12 to 14, a surgical suture cutting device 1 according to a fourth embodiment of the present invention has each of its positioning ribs 32 recessed to form a dent 321 along the edge interfacing the tweezers 90. Usually for a tweezers 90 of an ordinary thickness, it can be plugged into the chamber of the coupling section 30 easily. For a thicker tweezers 90a, the positioning ribs 32 are easier to deform and the opening of the chamber can be opened wider. As such, the thicker tweezers 90 can still be plugged into the coupling section 30 and be held there with greater reliability.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A surgical suture cutting device, comprising:
a body having two bulging sections extended from a front end and a notch formed between the bulging sections; and
a knife wrapped inside the body having a blade exposed in the notch;
wherein, with the blade and the bulging sections, the surgical suture cutting device can be operated with a single hand to perform both tissue pressing and suture cutting; since the blade is protected in the notch, the patient will not be mistakenly wounded; the bulging sections provide comfortable tissue pressing; and the knife can be simply manufactured with reduced production cost.

2. The surgical suture cutting device according to claim 1, wherein the body has a coupling section at a rear end for joining with a tweezers.

3. The surgical suture cutting device according to claim 2, wherein the coupling section has a chamber open to the rear end; and a plurality of positioning ribs are configured inside the chamber for locking the tweezers.

4. The surgical suture cutting device according to claim 3, wherein at least one of the positioning ribs is recessed to form a dent along an edge interfacing the tweezers.

5. The surgical suture cutting device according to claim 1, wherein an indentation is configured on a top side of the body.

6. The surgical suture cutting device according to claim 1, wherein a plurality of ribs are configured on a top side of the body.

7. The surgical suture cutting device according to claim 1, wherein the bulging sections are tilted to form an angle with the body.

8. The surgical suture cutting device according to claim 1, wherein the body is integrally formed with a tweezers.
